(19) United States
(12) Patent Application Publication
(10) Pub. No.: US 2016/0361564 A1
(43) Pub. Date: Dec. 15, 2016

(54) LIGHT THERAPY DEVICE

(71) Applicant: GCSOL Tech Co., Ltd., Taichung City (TW)

(72) Inventors: Jui-Fen PAI, Nantou County (TW); Yu-Wei YE, Taichung City (TW)

(21) Appl. No.: 14/845,297

(22) Filed: Sep. 4, 2015

(30) Foreign Application Priority Data
Jun. 11, 2015 (TW) 104118967

(51) Int. Cl. A61N 5/06; F21L 4/00; F21V 19/00; F21V 23/04; F21V 23/02; F21V 23/06

(52) U.S. Cl. CPC A61N 5/0619; F21V 23/023; F21V 23/06; F21V 19/003; F21V 23/0414; F21L 4/00; F21Y 2101/02; F21Y 2113/005

(57) ABSTRACT

A light therapy device includes a base, a circuit board, a power supply unit and a lid. The base includes a bottom surface, wherein a first accommodating space is formed in the base. The circuit board is disposed in the first accommodating space. The circuit board includes a lighting unit and an electrode set electrically connected to the lighting unit, in which the lighting unit emits light toward the bottom surface. The power supply unit is electrically connected to the electrode set. The lid has a second accommodating space for accommodating the power supply unit, in which the second accommodating space is eccentrically rotated relative to the first accommodating space for eccentrically rotating and switching the power supply unit to be conducted or non-conducted with the electrode set, thereby turning on or off the lighting unit.

### RELATED APPLICATIONS

The application claims priority to Taiwan Application Serial Number 104118967, filed on Jun. 11, 2015, which is herein incorporated by reference.

### BACKGROUND

Technical Field

The present disclosure relates to a light therapy device. More particularly, the present disclosure relates to a light therapy device that is thin and portable, and can select different light colors through rotational switching.

Description of Related Art

Acupuncture-Moxibustion is a pain relief method based on stimulating certain points of a human body. In a conventional acupuncture-moxibustion therapy, a tool (e.g. a metal needle) is inserted into an acupoint of a human body to dredge the channels and collaterals in a vessels network system of the human body, thereby eliminating obstruction of meridians to cure the vessels network system.

However, such an acupuncture-Moxibustion therapy may have potential dangerousness, thus a practitioner has to be professionally trained, and not everyone is able to practice it. Furthermore, its side effects such as fainting or wound infection also often occur.

Recently, a light therapy has been developed rapidly to replace the conventional Acupuncture-Moxibustion therapy for stimulating acupoints. The light therapy irradiates light to human's skin to induce a series of biochemical reactions to dredge the channels and collaterals in a vessels network system as the acupuncture-moxibustion therapy achieves.

However, the structure of the conventional light therapy device is too complicated and large to be used conveniently and portably. For example, a box-type light therapy device allows a user to enter therein to receive a full-body therapy. However, because the device is too big, it has to be set up in a specified location and cannot be moved freely. Another smaller light therapy device has been developed to overcome the aforementioned problem, but the operation thereof is too complicated and the size thereof is not small enough to be carried conveniently.

Accordingly, there is a need to develop a light therapy device which is portable and safe.

### SUMMARY

According to one aspect of the present disclosure, a light therapy device is provided. The light therapy device includes a base, a circuit board, a power supply unit and a lid. The base includes a bottom surface, in which a first accommodating space is formed in the base. The circuit board is disposed in the first accommodating space. The circuit board includes a lighting unit and an electrode set electrically connected to the lighting unit, in which the lighting unit emits a light toward the bottom surface. The power supply unit is electrically connected to the electrode set. The lid is rotatably received in the base and has a second accommodating space for accommodating the power supply unit. The second accommodating space is eccentrically rotated relative to the first accommodating space for eccentrically rotating and switching the power supply unit to be conducted or non-conducted with the electrode set, thereby turning on or off the lighting unit.

According to another aspect of the present disclosure, a light therapy device is provided. The light therapy device includes a base, a circuit board, a power supply unit and a lid. The base includes a bottom surface, wherein a first accommodating space is formed in the base. The circuit board is disposed in the first accommodating space. The circuit board includes a plurality of lighting units and an electrode set electrically connected to the lighting units, in which the lighting units emit lights toward the bottom surface. The power supply unit is electrically connected to the electrode set. The lid has a second accommodating space for accommodating the power supply unit, in which the second accommodating space is eccentrically rotated relative to the first accommodating space for eccentrically rotating and switching the power supply unit to be conducted or non-conducted with the electrode set, thereby sequentially turning on or off each of the lighting units.

### BRIEF DESCRIPTION OF THE DRAWINGS

The present disclosure can be more fully understood by reading the following detailed description of the embodiment, with reference made to the accompanying drawings as follows:

FIG. 1 is an exploded view showing a light therapy device according to one embodiment of the present disclosure;

FIG. 2 is an assembly view of the light therapy device of FIG. 1;

FIG. 3 is a cross-sectional view of the light therapy device of FIG. 1;

FIG. 4A is a schematic view showing that a power supply unit and an electrode set are not conducted;

FIG. 4B is a schematic view showing that the power supply unit and the electrode set are conducted;

FIG. 5A is a schematic view showing a light therapy device according to another embodiment of the present disclosure;

FIG. 5B is a schematic view showing an assembly of a lid and a base of FIG. 5A;

FIG. 6 is a schematic view showing another shape of a circuit board;

FIG. 7A is a schematic view showing an off status while multiple lighting units are used;

FIG. 7B is a schematic view showing a first lighting status while multiple lighting units are used;

FIG. 7C is a schematic view showing another off status while multiple lighting units are used;

FIG. 7D is a schematic view showing a second lighting status while multiple lighting units are used;

FIG. 8 is a schematic view showing a first application type of the light therapy device of the present disclosure; and

FIG. 9 is a schematic view showing a second application type of the light therapy device of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

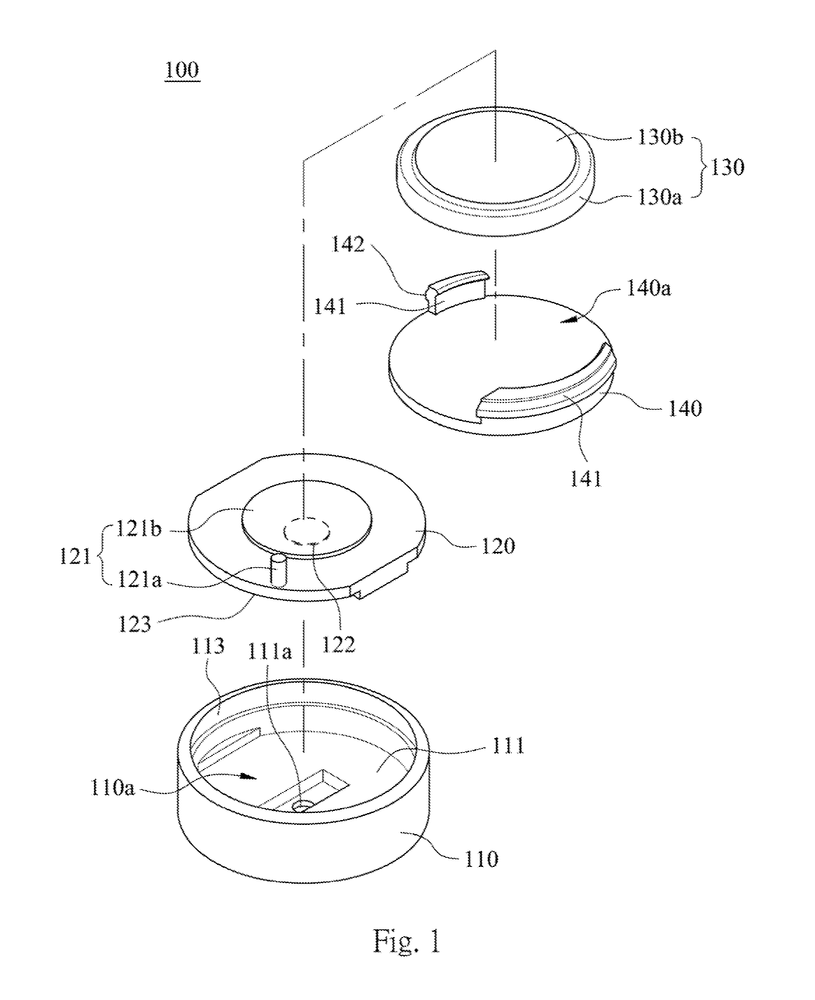

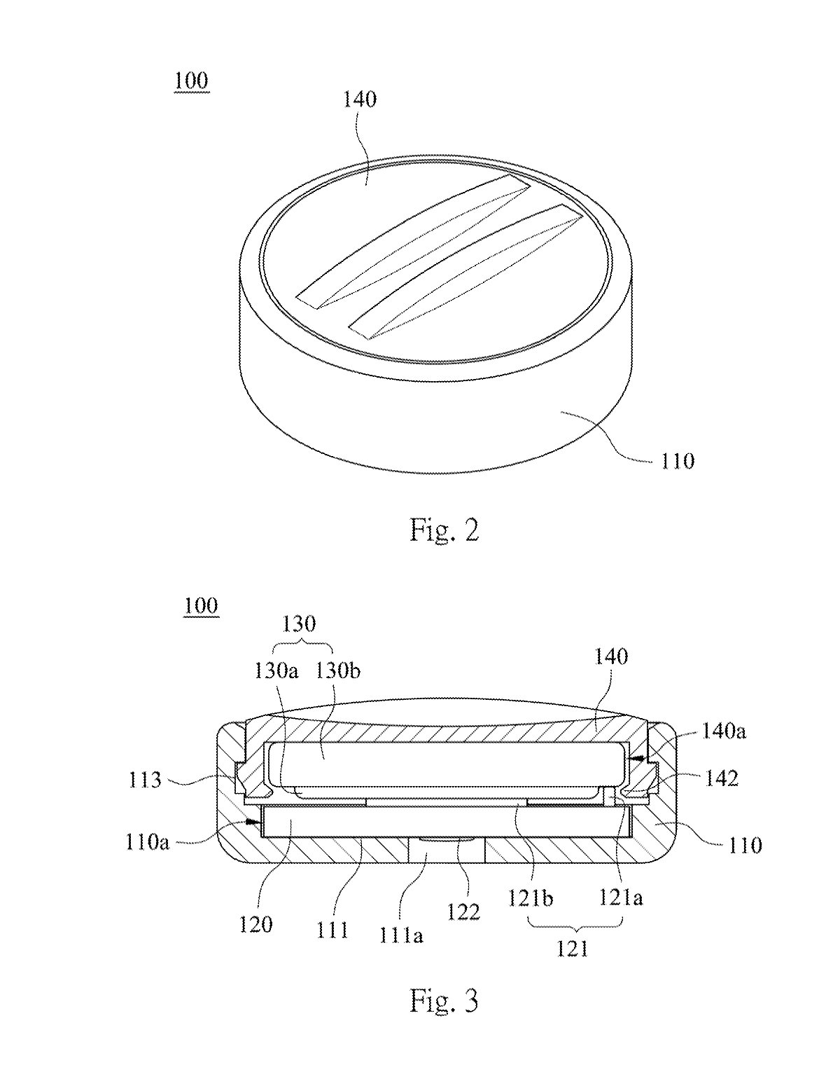

FIG. 1 is an exploded view showing a light therapy device 100 according to one embodiment of the present disclosure; FIG. 2 is an assembly view of the light therapy device 100 of FIG. 1; and FIG. 3 is a cross-sectional view of the light therapy device 100 of FIG. 1.

The light therapy device 100 includes a base 110, a circuit board 120, a power supply unit 130 and a lid 140.

The base 110 includes a bottom surface 111, and a first accommodating space 110a is formed in the base 110.

The circuit board 120 can be disposed in the first accommodating space 110a. A lighting unit 122 and an electrode set 121 are disposed on the circuit board 120. A driving circuit 123 can also be disposed on the circuit board 120 to switch the lighting modes of the lighting unit 122. The electrode set 121 includes a positive electrode member 121a and a negative electrode member 121b. The lighting unit 122, the electrode set 121 and the driving circuit 123 are electrically connected. The lighting unit 122 may be an inorganic light-emitting diode or an organic light-emitting diode. The lighting unit 122 emits light toward the bottom surface 111. A light through hole 111a is formed on the bottom surface 111. The lighting unit 122 emits light through the light through hole 111a to human skin for performing a light therapy.

A second accommodating space 140a is formed on the lid 140. The power supply unit 130 can be disposed in the second accommodating space 140a. The power supply unit includes a positive electrode 130a and a negative electrode 130b.

The lid 140 can be rotatably disposed in the base 110. In more detail, a groove 113 is formed in the base 110 and a track 141 is formed on the lid 140. The track 141 can be rotatably engaged with the groove 113.

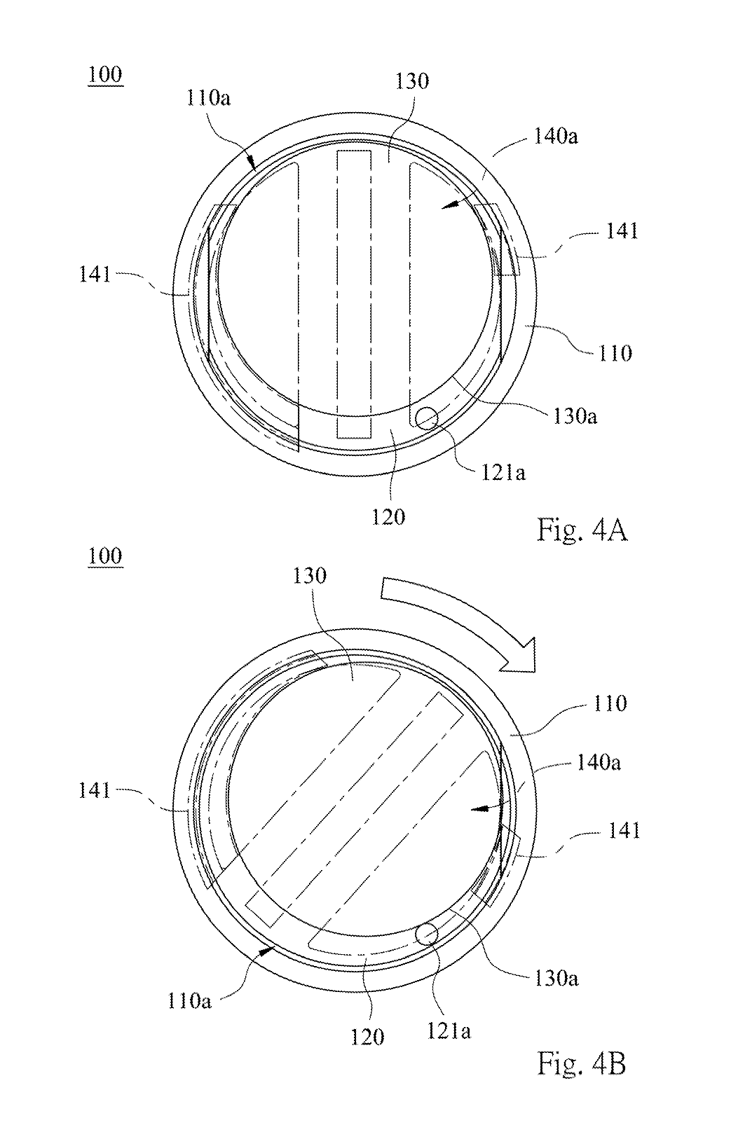

FIG. 4A is a schematic view showing that the power supply unit 130 and the electrode set 121 are not conducted; and FIG. 4B is a schematic view showing that the power supply unit 130 and the electrode set 121 are conducted.

When the lid 140 is engaged with the base 110, the positive electrode 130a of the power supply unit 130 can selectively contact or not contact the positive electrode member 121a of the electrode set 121, and the negative electrode 130b of the power supply unit 130 contacts the negative electrode member 121b of the electrode set 121. Accordingly, the power supply unit 130 and the electrode set 121 can be conducted or not conducted, and thus the lighting unit 122 can be turned on or turned off. In more detail, the second accommodating space 140a of the lid 140 can be rotated eccentrically relative to the first accommodating space 110a of the base 110. Since being fixedly disposed in the second accommodating space 140a of the lid 140, the power supply unit 130 can also be eccentrically rotated relative to the first accommodating space 101a while the lid 140 is rotated. Through the eccentric rotation, the power supply unit 130 and the electrode set 121 are conducted or not conducted, and thus the lighting unit 122 can be turned on or off.

In one example, the positive electrode member 121a of the electrode set 121 is pillar-shaped, and the shapes and positions of the positive electrode member 121a and the negative electrode member 121b of the electrode set can be constructed in accordance with the shapes and positions of the positive electrode 130a and the negative electrode 130b of the power supply unit 130. Therefore, when the power supply unit 130 is eccentrically rotated, the negative electrode 130b of the power supply unit 130 constantly contacts the negative electrode member 121b of the electrode set 121, and the positive electrode 130a of the power supply unit 130 selectively contacts the positive electrode member 121a of the electrode set 121. Thus, the conducting or non-conducting status between the power supply unit 130 and the electrode set 121 of the circuit board 120 can be selectively determined.

The aforementioned power supply unit 130 can be a thin-film battery, a fuel battery or a coin battery for achieving compact size and portability.

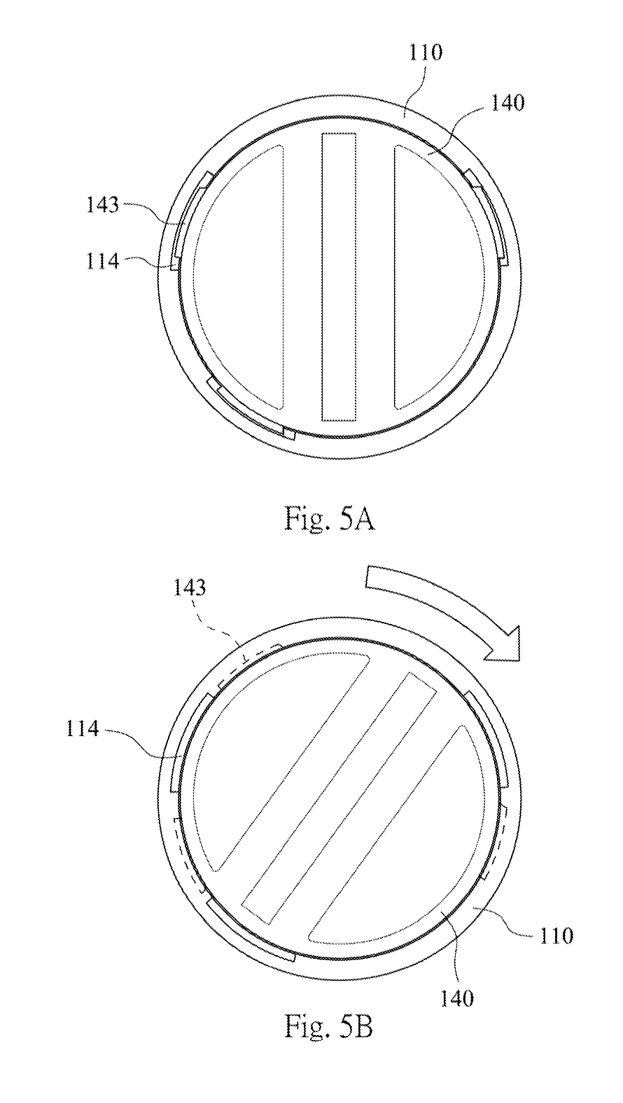

FIG. 5A is a schematic view showing a light therapy device 100 according to another embodiment of the present disclosure; and FIG. 5B is a schematic view showing an assembly of a lid 140 and a base 110 of FIG. 5A. The aforementioned light therapy device 100 may have various types. For example, in FIGS. 1-3, a barb 142 is formed on a track 141, and thus the lid 140 and the base 110 cannot be detached when being engaged with each other. In this situation, a single-use light therapy device 100 is formed, in which the power supply unit 130 cannot be replaced when being exhausted. In another example, as in FIGS. 5A and 5B, open grooves 114 are formed on the base 110, and engaging portions 143 corresponding to the open grooves 114 are formed on the lid 140, and the barb 142 is not formed on the track 141 of the lid 140. The lid 140 can be engaged with the base 114 by placing the engaging portion 143 into the open groove 114 and rotating the engaging portion 143 clockwise. In this situation, when the power supply unit 130 is exhausted, the lid 140 and the base 110 can be separated by rotating the engaging portion 143 counterclockwise. Therefore, the power supply unit 130 is removable and can be replaced with a new one, and thus the light therapy device 100 is reusable.

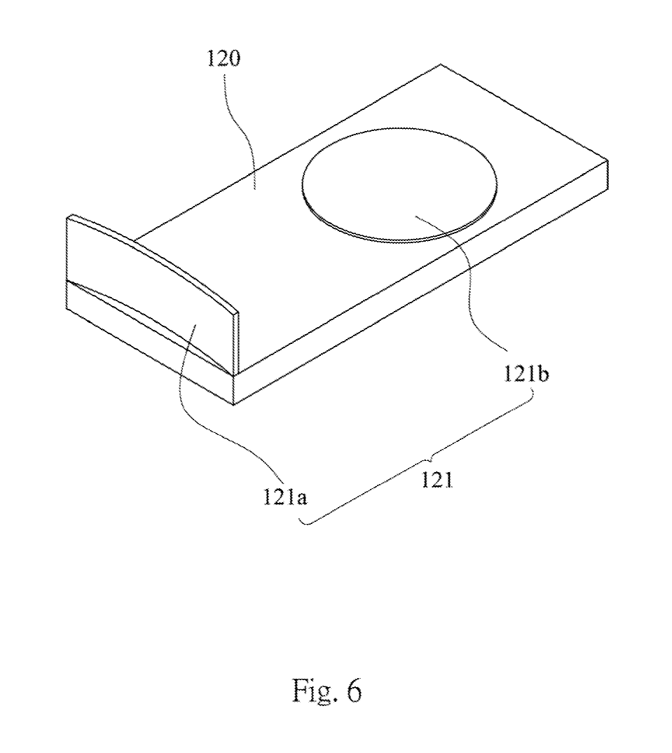

FIG. 6 is a schematic view showing another shape of the circuit board 120. In the present disclosure, the shape of the circuit board 120 is not limited thereto as long as the following conditions are satisfied. Firstly, the circuit board 120 should be able to be accommodated in the first accommodating space 110a of the base 110. Secondly, when the power unit 130 is eccentrically rotated, the negative electrode 130b of the power supply unit 130 should constantly contact the negative electrode member 121b of the circuit board 120, and the positive electrode 130a of the power supply unit 130 should selectively contact the positive electrode member 121a of the circuit board 120 for turning on/off the lighting unit 122. As shown in FIG. 6, the circuit board 120 can be a rectangle, and the positive electrode member 121a of the circuit board 120 can be a curved plate. The shape of the positive electrode member 121a and the shape of the circuit board 120 in FIG. 6 can also satisfy the aforementioned conditions when the power supply unit 130 is eccentrically rotated.

In other words, the positive electrode member 121a and the negative electrode member 121b are activated in accordance with the electrodes of the power supply unit 130. For example, if the power supply unit 130 is a coin battery, the positive electrode 130a is located in its side, and the negative electrode 130b is located in its center. Therefore, the negative electrode 130b is configured to be constantly conducted, and the positive electrode 130a is configured to be selectively conducted. In another example, the positions of the positive electrode 130a and the negative electrode 130b may be exchanged. In this situation, the positive electrode 130a is constantly conducted, and the negative electrode 130b is selectively conducted.

The number of the lighting units 122 in the present disclosure is also not limited thereto. Multiple lighting units 122 may possibly be used for generating multiple light colors through the driving circuit 123. The lighting units 122 may emit light in the same color or in different colors. When the lighting units 122 emit light in different colors, different types of therapy effect can be achieved; and when the lighting units 122 emit lights in the same color, different light intensity and lighting levels can be obtained for achieving different therapy effects.

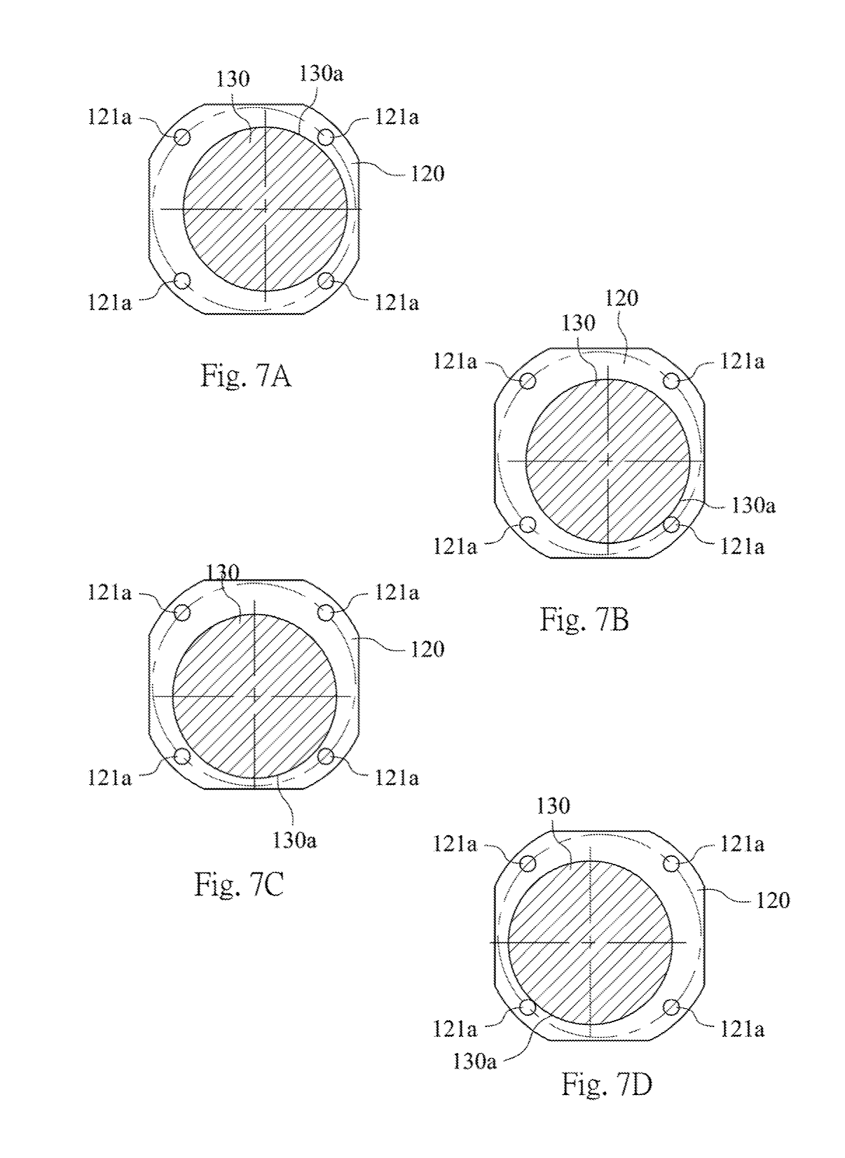

FIG. 7A is a schematic view showing an off status while multiple lighting units 122 are used; FIG. 7B is a schematic view showing a first lighting status while multiple lighting units 122 are used; FIG. 7C is a schematic view showing another off status while multiple lighting units 122 are used; and FIG. 7D is a schematic view showing a second lighting status while multiple lighting units 122 are used.

In FIGS. 7A to 7D, the electrode set 121 of the circuit board 120 includes a negative electrode member 121b and four positive electrode members 121a. As previously described, the power supply unit 130 is eccentrically rotated, and the positive electrode members 121a sequentially contacts or do not contact each of the positive electrode members 121a, and the negative electrode 130b of the power supply unit 130 constantly contacts the negative electrode member 121b of the electrode set 121. Therefore, the lighting units 122 can be turned on or off sequentially. When the lighting units 122 emit light in different colors, as shown in FIG. 7A, the power supply unit 130 is eccentrically rotated, and thus the positive electrode 130a thereof will not contact any of the positive electrodes and is not conducted, and the light therapy device 100 is in an off status. Then, in FIG. 7B, the positive electrode 130a of the power supply unit 130 contacts one of the positive electrodes members 121a owing to the power supply unit 130 is eccentrically rotated. Therefore, the light therapy device 100 is in a first on status and a first light color is emitted. Then, in FIG. 7C, the power supply unit 130 is eccentrically rotated again to enable the positive 130a thereof to not contact any positive electrode member 121a, and thus the light therapy device 100 is in an off status again. Then, in FIG. 7D, the power supply unit 130 is eccentrically rotated again, and thus the positive electrode 130a thereof contact another positive electrode member 121, and the light therapy device 100 is in a second on status, and a second light color is emitted. By using the aforementioned method, the positive electrode 130a of the power supply unit 130 sequentially contact each of the positive electrode members 121a, and thus a conducting/non-conducting cycle is formed, and the lighting units 122 can sequentially emit light in different colors or can emit light in the same color with different light intensity or lighting levels. When one single lighting unit 122 is used, different light modes can also be achieved through different designs of the driving circuit 123. Through the driving circuit 123, the light therapy device 100 is capable of having different light intensities and lighting levels of single color light.

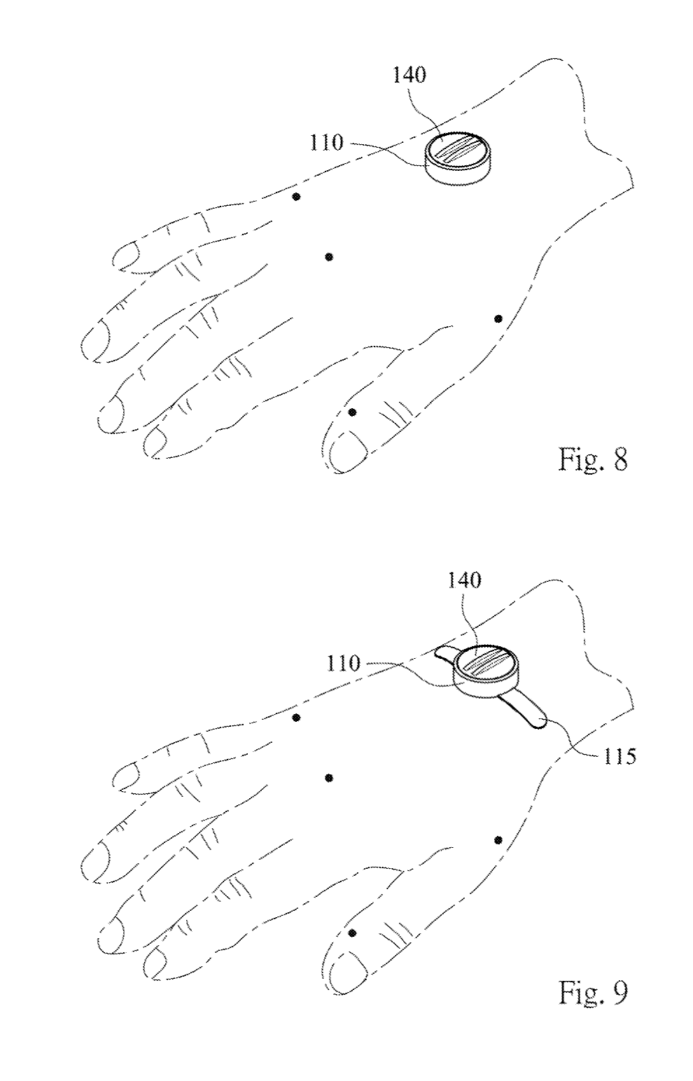

FIG. 8 is a schematic view showing a first application type of the light therapy device 100 of the present disclosure; and FIG. 9 is a schematic view showing a second application type of the light therapy device 100 of the present disclosure.

In one example, as shown in FIG. 8, the base 110 of the light therapy device 100 is attached to human skin, and the lighting unit 122 emits light through the light through hole 111a formed on the bottom surface 111 of the base 110 to the human skin. The lighting modes of the lighting unit 122 can be switched by rotating the lid 140. In another example, as shown in FIG. 9, the base 110 has an extension portion 115 to attach human skin with a larger area, and thus the light therapy device 100 can be fixed more firmly. The extension portion 115 can be integrally formed while the base 110 is manufactured, or can be additionally formed, but its manufacturing type is not limited thereto.

In sum, the light therapy device 100 has the advantages of compact size, portability and simple structure. Therefore, the manufacturing cost is low and is suitable for mass production. Furthermore, the light therapy device 100 also has the advantages of high durability and long operation life.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure lid modifications and variations of this disclosure provided they fall within the scope of the following claims.

What is claimed is:

1. A light therapy device, comprising:
a base comprising a bottom surface, wherein a first accommodating space is formed in the base;
a circuit board disposed in the first accommodating space, the circuit board comprising a lighting unit and an electrode set electrically connected to the lighting unit, wherein the lighting unit emits a light toward the bottom surface;
a power supply unit electrically connected to the electrode set; and
a lid on which a second accommodating space is formed for accommodating the power supply unit, wherein the lid rotatably received in the base, and the second accommodating space is eccentrically rotated relative to the first accommodating space for eccentrically rotating and switching the power supply unit to be conducted or non-conducted with the electrode set, thereby turning on or off the lighting unit.

2. The light therapy device of claim 1, wherein a light through hole is formed on the bottom surface, and light emitted from the lighting unit is emitted through the light through hole to human skin.

3. The light therapy device of claim 1, wherein the circuit board comprises a driving circuit, and the driving circuit is electrically connected to the electrode set, and the lighting unit is driven by the driving circuit to switch a lighting mode of the lighting unit.

4. The light therapy device of claim 1, wherein the base comprises a groove, and the lid comprises a track, and the track is rotatably engaged with the groove.

5. The light therapy device of claim 4, wherein the track comprises a barb, and the barb is formed on the track to enable the lid and the base to be undetachable after being engaged.

6. The light therapy device of claim 4, wherein the base comprises a plurality of open grooves, and the lid comprises a plurality of engaging portions corresponding to the open grooves, and the lid and the base are dismantable through the open grooves and the engaging portions when being engaged with each other.

7. The light therapy device of claim 1, wherein the power supply unit is a thin-film battery, a fuel battery or a coin battery.

8. The light therapy device of claim 7, wherein the electrode set comprises a positive electrode member and a negative electrode member, and the power supply unit comprises a positive electrode and a negative electrode, and the power supply unit is eccentrically rotated to enable the positive electrode of the power supply unit to selectively contact or not contact the positive electrode member of the electrode set, and the negative electrode of the power supply unit to contact the negative electrode member of the electrode set.

9. The light therapy device of claim 8, wherein the positive electrode member of the electrode set is pillar-shaped.

10. The light therapy device of claim 7, wherein the electrode set comprises a plurality of positive electrode members and a negative electrode member, and the power supply unit comprises a positive electrode and a negative electrode, and the power supply unit is eccentrically rotated to enable the positive electrode of the power supply unit to selectively and sequentially contact the positive electrode members of the electrode set, and the negative electrode of the power supply unit to contact the negative electrode member of the electrode set.

11. The light therapy device of claim 1, wherein the lighting unit is an inorganic light-emitting diode or an organic light-emitting diode.

12. The light therapy device of claim 1, wherein the base comprises an extension portion attachable to human skin.

13. A light therapy device, comprising:
a base comprising a bottom surface, wherein a first accommodating space is formed in the base;
a circuit board disposed in the first accommodating space, the circuit board comprising a plurality of lighting units and an electrode set electrically connected to the lighting units, wherein the lighting units emit lights toward the bottom surface;
a power supply unit electrically connected to the electrode set; and
a lid having a second accommodating space for accommodating the power supply unit, wherein the second accommodating space is eccentrically rotated relative to the first accommodating space for eccentrically rotating and switching the power supply unit to be conducted or non-conducted with the electrode set, thereby sequentially turning on or off each of the lighting units.

14. The light therapy device of claim 13, wherein the electrode set comprises a plurality of positive electrode members and a negative electrode member, and the power supply unit comprises a positive electrode and a negative electrode, and the power supply unit is eccentrically rotated to enable the positive electrodes of the power supply unit to sequentially contact the positive electrode member of the electrode set, and the negative electrode of the power supply unit to contact the negative electrode member of the electrode set.

15. The light therapy device of claim 13, wherein the lighting units are sequentially switched to emit light in different colors.

16. The light therapy device of claim 13, wherein the lighting units emit light in the same color.

17. The light therapy device of claim 13, wherein each of the lighting units is an inorganic light-emitting diode or an organic light-emitting diode.
